(19) United States
(12) Patent Application Publication
(10) Pub. No.: US 2016/0287751 A1
(43) Pub. Date: Oct. 6, 2016

(54) INJECTABLE AMNIOTIC MEMBRANE TISSUE GRAFT

(71) Applicant: Applied Biologics, LLC, Scottsdale, AZ (US)

(72) Inventor: Edward Britt, Scottsdale, AZ (US)

(21) Appl. No.: 14/986,188

(22) Filed: Dec. 31, 2015

Related U.S. Application Data
(60) Provisional application No. 62/098,999, filed on Dec. 31, 2014.

(51) Int. Cl. A61L 27/36; A61K 35/50

(52) U.S. Cl. CPC A61L 27/3633; A61K 35/50

(57) ABSTRACT

The invention relates to an injectable amniotic membrane tissue graft product for percutaneous, minimally invasive, surgical, and topical therapy of injury and disease and methods of applying such tissue grafts to a subject at a particular location. The injectable preparations maximize and standardize available quantities of non-cellular biological compounds to enhance therapeutic efficacy. The tissue graft preparations are semi-viscous fluids with standardized properties which may be intraoperatively transplanted at the recipient site using a needless syringe, by non-operative percutaneous injection through a hypodermic needle, or direct topical application to open cutaneous wounds and external soft tissue defects.

### CROSS REFERENCE TO RELATED APPLICATION

This application claims from the benefit of U.S. Provisional Application No. 62/098,999, filed Dec. 31, 2014, the contents of which are incorporated entirely herein by reference.

### BACKGROUND OF THE INVENTION

1. Technical Field

This invention relates to an injectable tissue graft comprising a suspension of amniotic membrane particles with standardized properties. In particular, embodiments of the invention relate to a mammalian liquid amniotic membrane tissue graft product with preselected properties that are suitable for administration to a particular site in or on a subject, and methods of applying such a tissue graft.

2. State of the Art

Amniotic membrane, specifically human amniotic membrane, has been used in surgery for over one hundred years. The amnion's interstitial matrix and cellular components contain a complex biologic soup of growth factors, inflammatory mediators, immuno-modulators, and other active biomolecules. Additionally, amniotic membrane is rich in embryonic stem cells containing high concentrations of these biologically active substances.

Amniotic membrane is used in a variety of surgical procedures as an adjunct to healing, and to minimize formation of scar tissue and adhesions. Hydration of the amniotic membrane may be fully or partially maintained; or, alternatively, the amniotic membrane may be dried prior to packaging, sterilization, and storage. Some preparations reconstitute the dried amniotic membrane prior to patient use by using a tissue preservative solution prior to the packaging and sterilization for storage. A sheet of amniotic membrane, whether fresh or reconstituted, however, is suitable only for cutaneous or intraoperative placement on/in the recipient tissue bed.

Accordingly, what is needed is an amniotic membrane tissue graft product which is liquid of a sufficiently low viscosity for percutaneous injection into the recipient host tissue bed.

Citation of documents herein is not an admission by the applicant that any is pertinent prior art. Stated dates or representation of the contents of any document is based on the information available to the applicant and does not constitute any admission of the correctness of the dates or contents of any document.

### DISCLOSURE OF EMBODIMENTS OF THE INVENTION

It will be appreciated by practitioners in the art that certain tissue graft properties are more desirable or well-suited for various applications. For instance, properties such as viscosity, particle size, and concentration can affect how well a particular tissue graft is suited for administration at a soft tissue or hard tissue site on a patient in need of a tissue graft. Furthermore, practitioners in the art will appreciate that the lack of available tissue grafts with known standardized properties presents a tremendous disadvantage in terms of consistently identifying and obtaining tissue grafts suited for a particular application. This is especially true in the case of tissue grafts that include amniotic membrane particles as a major component, due to the variability in available amniotic membrane tissue properties and the resulting variability in tissue grafts produced from such components.

The advantage of having an available source of standardized tissue grafts with known properties is the consistency that a tissue graft with such properties provides to the practitioner and the subject in need of the tissue graft. For example, one can select a tissue graft or a set of tissue grafts from a source with the knowledge that properties such as viscosity, amniotic membrane particle size, and/or amniotic membrane particle concentration are consistent between individual tissue grafts. This presents a huge advantage to a practitioner and/or subject in need of a tissue graft or multiple tissue grafts with specific properties. The ability to obtain tissue grafts with standardized properties is particularly advantageous in instances where the tissue graft is selected for application to a subject at a particular location. For instance, a tissue graft with particular amniotic membrane particle concentration, amniotic membrane particle size, and viscosity values may be better suited for application to a hard tissue (i.e., bone) site than a soft tissue (e.g., skin or muscle) site in or on a patient. The availability of a source of standardized tissue grafts with particular known properties would be highly advantageous in such a situation since one could consistently procure one or more tissue grafts with optimal properties for application to a particular site in or on a subject.

For instance, in general, in the case of application of an amniotic membrane tissue graft to soft tissue, it is advantageous to use tissue grafts with a relatively low concentration of amniotic membrane particles but with high viscosity and relatively large amniotic membrane particles. On the other hand, in general, in the case of application of an amniotic membrane tissue graft to hard tissue such as bone, it is advantageous to use tissue grafts with a relatively high concentration of amniotic membrane particles but with low viscosity and smaller amniotic membrane particles. In some instances, it may be advantageous to include hydroxyapatite in tissue grafts intended for application to bone. However, at present, no such standardized tissue grafts with particular properties suited to particular applications or methods of applying such tissue grafts is known in the art.

The current invention provides a solution to these problems by providing standardized tissue grafts and sets of standardized tissue grafts that include a suspension of amniotic membrane particles with pre-selected properties that are advantageous to performing specific procedures on a subject. Furthermore, the invention provides methods of applying a tissue graft with pre-selected properties to a particular location in or on a subject, such that the properties of the tissue graft (e.g., amniotic membrane particle size, viscosity, and concentration) are selected to enhance applicability of the tissue graft at that location.

The invention includes methods of applying to a subject a tissue graft comprising a suspension of amniotic membrane particles. Methods of the invention include applying a tissue graft with specialized properties designed for application to specific locations in or on a patient's body. For example, methods of the invention include applying a tissue graft to a location in or on the subject where the suspension of amniotic membrane particles is designed with selected properties such as amniotic membrane particle concentration, amniotic membrane particle size, or suspension viscosity. For example, methods of the invention include applying a tissue graft to a location in or on the subject, wherein the suspension has a concentration, a viscosity or an average amniotic membrane particle size selected to enhance the applicability of the tissue graft at that location. In some embodiments, methods of the invention include applying a tissue graft comprising a suspension of amniotic membrane particles to a location of, in, or on the subject, wherein the suspension has a quantity selected from the group of quantities that includes a concentration, a viscosity, and an average amniotic membrane particle size selected to enhance the applicability of the tissue graft at the location.

In some embodiments the invention includes methods for applying tissue grafts to various locations in or on a subject. For instance, in some embodiments the invention includes methods of applying a tissue graft to a location in on a subject, where the location comprises bone or soft tissue. Soft tissue may include any soft tissue in or on the subject. For instance, in some embodiments the soft tissue is selected from the group consisting of muscle, tendon, ligament, and skin. Furthermore, methods of the invention include methods for applying tissue grafts to any location in or on a subject, such as, for instance, cartilage, epithelial tissue, connective tissue, muscle tissue, or nervous tissue. In some instances, the invention includes methods of applying a tissue graft where the amniotic membrane suspension further includes hydroxyapatite. The addition of hydroxyapatite is especially useful in methods that entail the application of tissue graft to bone.

In some embodiments of the invention, the method of applying to a subject a tissue graft comprising a suspension of amniotic membrane particles includes a tissue graft that is substantially free of chorion particles. This is especially advantageous in methods of applying a tissue graft to a site where the inclusion of chorion particles could elicit a strong antigenic response.

Some embodiments of the invention include methods of applying to a subject a tissue graft comprising a suspension of amniotic membrane particles where the average amniotic membrane particle size is preselected. For instance, in some embodiments the invention includes methods of applying to a subject a tissue graft comprising a suspension of amniotic membrane particles where the average amniotic membrane particle size is less than 10 microns, less than 20 microns, less than 30 microns, less than 40 microns, less than 50 microns, less than 60 microns, less than 70 microns, less than 75 microns, less than 90 microns, or less than 100 microns. In some embodiments the invention includes methods of applying to a subject a tissue graft comprising a suspension of amniotic membrane particles where the average amniotic membrane particle size is at least 10 microns, at least 20 microns, at least 30 microns, at least 40 microns, at least 50 microns, at least 60 microns, at least 75 microns, at least 80 microns, at least 90 microns, at least 100 microns, at least 110 microns, at least 120 microns, at least 130 microns, at least 140 microns, or at least 150 microns. In some embodiments the invention includes methods of applying to a subject a tissue graft comprising a suspension of amniotic membrane particles where the average amniotic membrane particle size is 10-25 microns, 25-50 microns, 50-75 microns, 75-100 microns, 100-125 microns, or 125-150 microns.

The invention also includes methods of applying to a subject a tissue graft comprising a suspension of amniotic membrane particles where the concentration of amniotic membrane particles in the suspension is preselected or of a standard concentration. For instance, in some embodiments the invention includes methods of applying to a subject a tissue graft comprising a suspension of amniotic membrane particles where the concentration of amniotic membrane particles in the suspension is 0.01-0.10 mg/ml, 0.10-1 mg/ml, 1-10 mg/ml, 1-5 mg/ml, 0.01-0.05 g/ml, 0.05-0.10 g/ml, 0.10-0.15 g/ml, 0.10-0.20 g/ml, 0.15-0.20 g/ml, 0.20-0.25 g/ml, 0.25-0.30 g/ml, 0.30-0.35 g/ml, 0.35-0.40 g/ml, 0.40-0.45 g/ml, or 0.45-0.50 g/ml. In some embodiments the invention includes methods of applying to a subject a tissue graft comprising a suspension of amniotic membrane particles where the concentration of amniotic membrane particles in the suspension is between about 0.01-0.10 mg/ml, between about 0.10-1 mg/ml, between about 1-10 mg/ml, between about 1-5 mg/ml, between about 0.01-0.05 g/ml, between about 0.05-0.10 g/ml, between about 0.10-0.15 g/ml, between about 0.10-0.20 g/ml, between about 0.15-0.20 g/ml, between about 0.20-0.25 g/ml, between about 0.25-0.30 g/ml, between about 0.30-0.35 g/ml, between about 0.35-0.40 g/ml, between about 0.40-0.45 g/ml, or between about 0.45-0.50 g/ml. In some embodiments the invention includes methods of applying to a subject a tissue graft comprising a suspension of amniotic membrane particles where the concentration of amniotic membrane particles in the suspension is about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, about 5 mg/ml, about 10 mg/ml, about 20 mg/ml, about 30 mg/ml, about 40 mg/ml, about 50 mg/ml, about 60 mg/ml, about 70 mg/ml, about 80 mg/ml, about 90 mg/ml, or about 100 mg/ml. In some embodiments the invention includes methods of applying to a subject a tissue graft comprising a suspension of amniotic membrane particles where the concentration of amniotic membrane particles in the suspension is greater than 0.01 mg/ml, greater than 0.1 mg/ml, greater than 1 mg/ml, greater than 5 mg/ml, greater than 10 mg/ml, greater than 20 mg/ml, greater than 30 mg/ml, greater than 40 mg/ml, greater than 50 mg/ml, greater than 60 mg/ml, greater than 70 mg/ml, greater than 80 mg/ml, greater than 90 mg/ml, or greater than 100 mg/ml. In some embodiments the invention includes methods of applying to a subject a tissue graft comprising a suspension of amniotic membrane particles where the concentration of amniotic membrane particles in the suspension is at least 0.01 mg/ml, at least 0.1 mg/ml, at least 1 mg/ml, at least 5 mg/ml, at least 0.01 g/ml, at least 0.05 g/ml, at least 0.10 g/ml, at least 0.20 g/ml, at least 0.30 g/ml, at least 0.40 g/ml, or at least 0.50 g/ml. In some embodiments the invention includes methods of applying to a subject a tissue graft comprising a suspension of amniotic membrane particles where the concentration of amniotic membrane particles in the suspension is less than 0.01 mg/ml, less than 0.1 mg/ml, less than 1 mg/ml, less than 5 mg/ml, less than 10 mg/ml, less than 20 mg/ml, less than 30 mg/ml, less than 40 mg/ml, less than 0.05 g/ml, less than 0.10 g/ml, less than 0.20 g/ml, less than 0.30 g/ml, less than 0.40 g/ml, less than 0.50 g/ml, less than 0.75 g/ml, or less than 1.00 g/ml.

Furthermore, the invention includes methods of applying a tissue graft comprising a suspension of amniotic membrane particles to a subject where the viscosity of the tissue graft suspension is preselected. For instance, the tissue graft may be a tissue graft of relatively low, medium, or high viscosity depending on the intended application.

In addition to methods of applying a tissue graft, the invention includes tissue grafts with preselected properties that may be selected for a particular applications to which they are best suited. For instance, embodiments of the invention include tissue grafts comprising a suspension of amniotic membrane particles with various amniotic membrane particle size values, amniotic membrane particle concentration values, and viscosity values. The invention includes tissue grafts containing suspensions with various combinations of these characteristics which may be better suited for different applications, for instance, application to different sites in or on a patient, for instance, application to bone or soft tissue.

Some embodiments of the invention include a tissue graft comprising a suspension of amniotic membrane particles where the average amniotic membrane particle size is preselected. For instance, in some embodiments the invention includes a tissue graft comprising a suspension of amniotic membrane particles where the average amniotic membrane particle size is less than 10 microns, less than 20 microns, less than 30 microns, less than 40 microns, less than 50 microns, less than 60 microns, less than 70 microns, less than 75 microns, less than 90 microns, or less than 100 microns. In some embodiments the invention includes a tissue graft comprising a suspension of amniotic membrane particles where the average amniotic membrane particle size is at least 10 microns, at least 20 microns, at least 30 microns, at least 40 microns, at least 50 microns, at least 60 microns, at least 75 microns, at least 80 microns, at least 90 microns, at least 100 microns, at least 110 microns, at least 120 microns, at least 130 microns, at least 140 microns, or at least 150 microns. In some embodiments the invention includes a tissue graft comprising a suspension of amniotic membrane particles where the average amniotic membrane particle size is 10-25 microns, 25-50 microns, 50-75 microns, 75-100 microns, 100-125 microns, or 125-150 microns.

The invention also includes tissue grafts comprising a suspension of amniotic membrane particles where the concentration of amniotic membrane particles in the suspension is preselected. For instance, in some embodiments the invention includes a tissue graft comprising a suspension of amniotic membrane particles of a standard concentration, where the concentration of amniotic membrane particles in the suspension is 0.01-0.10 mg/ml, 0.10-1 mg/ml, 1-10 mg/ml, 1-5 mg/ml, 0.01-0.05 g/ml, 0.05-0.10 g/ml, 0.10-0.15 g/ml, 0.10-0.20 g/ml, 0.15-0.20 g/ml, 0.20-0.25 g/ml, 0.25-0.30 g/ml, 0.30-0.35 g/ml, 0.35-0.40 g/ml, 0.40-0.45 g/ml, or 0.45-0.50 g/ml. In some embodiments the invention includes a tissue graft comprising a suspension of amniotic membrane particles where the concentration of amniotic membrane particles in the suspension is between about 0.01-0.10 mg/ml, between about 0.10-1 mg/ml, between about 1-10 mg/ml, between about 1-5 mg/ml, between about 0.01-0.05 g/ml, between about 0.05-0.10 g/ml, between about 0.10-0.15 g/ml, between about 0.10-0.20 g/ml, between about 0.15-0.20 g/ml, between about 0.20-0.25 g/ml, between about 0.25-0.30 g/ml, between about 0.30-0.35 g/ml, between about 0.35-0.40 g/ml, between about 0.40-0.45 g/ml, or between about 0.45-0.50 g/ml. In some embodiments, the invention includes a tissue graft where the concentration of amniotic membrane particles is about 0.1 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, about 1 mg/ml, about 5 mg/ml, about 10 mg/ml, about 20 mg/ml, about 30 mg/ml, about 40 mg/ml, about 50 mg/ml, about 60 mg/ml, about 70 mg/ml, about 80 mg/ml, about 90 mg/ml, or about 100 mg/ml. In some embodiments, the invention includes a tissue graft where the concentration of amniotic membrane particles is greater than 0.01 mg/ml, greater than 0.1 mg/ml, greater than 1 mg/ml, greater than 5 mg/ml, greater than 10 mg/ml, greater than 20 mg/ml, greater than 30 mg/ml, greater than 40 mg/ml, greater than 50 mg/ml, greater than 60 mg/ml, greater than 70 mg/ml, greater than 80 mg/ml, greater than 90 mg/ml, or greater than 100 mg/ml. In some embodiments the invention includes a tissue graft comprising a suspension of amniotic membrane particles where the concentration of amniotic membrane particles in the suspension is at least 0.01 mg/ml, at least 0.1 mg/ml, at least 1 mg/ml, at least 5 mg/ml, at least 0.01 g/ml, at least 0.05 g/ml, at least 0.10 g/ml, at least 0.20 g/ml, at least 0.30 g/ml, at least 0.40 g/ml, or at least 0.50 g/ml. In some embodiments the invention includes a tissue graft comprising a suspension of amniotic membrane particles where the concentration of amniotic membrane particles in the suspension is less than 0.01 mg/ml, less than 0.1 mg/ml, less than 1 mg/ml, less than 5 mg/ml, less than 10 mg/ml, less than 20 mg/ml, less than 30 mg/ml, less than 40 mg/ml, less than 0.05 g/ml, less than 0.10 g/ml, less than 0.20 g/ml, less than 0.30 g/ml, less than 0.40 g/ml, less than 0.50 g/ml, less than 0.75 g/ml, or less than 1.00 g/ml.

In a particular embodiment, the invention includes a tissue graft comprising a suspension of amniotic membrane particles, wherein the average amniotic membrane particle size is less than 75 microns and the concentration of amniotic membrane particles in the suspension is between 0.15 g/mL and 0.20 g/mL. In another specific embodiment, the invention includes a tissue graft comprising a suspension of amniotic membrane particles, wherein the average amniotic membrane particle size is between about 75 microns and about 100 microns and the concentration of amniotic membrane particles in the suspension is between 0.05 g/mL and 0.10 g/mL.

Furthermore, the invention includes tissue grafts comprising a suspension of amniotic membrane particles where the viscosity of the tissue graft suspension is preselected. For instance, the tissue graft may be a tissue graft of relatively low, medium, or high viscosity depending on the intended application. In some embodiments, the tissue graft may also include a thickening agent, for example, but not limited to, propylene glycol alginate, sodium-alginate, a polysaccharide thickening agent, a gelling agent, a thixotropic agent, a phase changing agent, hyaluronic acid, collagen, a thrombin gel, a fibrin gel, a fibrin glue, a gel forming agent (such as, for example, Pluronic®), dextran, carboxymethyl cellulose, polyethylene glycol, carbapol, liposomes, prohposomes, glycerol, starch, carbohydrates, povidone, polyethylene oxide, or polyvinyl alcohol.

In a particular embodiment, the invention includes a tissue graft comprising a suspension of amniotic membrane particles, wherein the average amniotic membrane particle size is less than 75 microns and the concentration of amniotic membrane particles in the suspension is 0.15-0.20 g/mL. In another particular embodiment, the invention includes a tissue graft comprising a suspension of amniotic particles, wherein the average amniotic membrane particle size is 75-100 microns and the concentration of amniotic membrane particles in the suspension is 0.05-0.10 g/mL.

In some embodiments, tissue grafts of the invention contain an amniotic membrane suspension that also includes hydroxyapatite, which is particularly advantageous for applications that involve application of the tissue graft to bone. In some embodiments, tissue grafts of the invention are substantially free of chorion particles. The absence of chorion particles in the tissue graft is particularly useful for avoiding an antigenic response from the subject.

Also disclosed is a fluidized amniotic membrane tissue graft product, including a method of forming same, comprising milled amniotic membrane reconstituted with a fluid wherein the resulting tissue graft has an appropriate viscosity for use as an injectable amniotic membrane tissue graft.

Also disclosed is a tissue graft comprising a fluid and an amniotic membrane, wherein the amniotic membrane is suspended within the fluid.

In some embodiments, the amniotic membrane is dried prior to suspension within the fluid. In some embodiments, the fluid comprises an isotonic electrolyte solution. In some embodiments, the fluid comprises a cryoprotectant. In some embodiments, the fluid comprises an isotonic electrolyte solution and a cryoprotectant. In some embodiments, the amniotic membrane comprises a mammalian amnion.

Disclosed is a method of forming a tissue graft comprising the steps of milling an amnion and suspending the milled amnion with a quantity of fluid to form an injectable amniotic membrane tissue graft.

In some embodiments, the method further comprises drying an amnion prior to milling the amnion. In some embodiments, the fluid comprises an isotonic electrolyte solution. In some embodiments, the fluid comprises a cryoprotectant. In some embodiments, the fluid comprises an isotonic electrolyte solution and a cryoprotectant. In some embodiments, the fluid comprises an aqueous solution of dimethylsulfoxide at a concentration of between 5% and 90% by weight. In some embodiments, the method further comprises lyophilizing the injectable amniotic membrane tissue graft.

The foregoing and other features and advantages of the present invention will be apparent from the following more detailed description of the particular embodiments of the invention, as illustrated in the accompanying drawings.

### BRIEF DESCRIPTION OF THE DRAWINGS

FIG. 1 is a flow chart showing steps of method 100 of forming an injectable amniotic membrane tissue graft;

FIG. 2 is a flow chart showing steps of method 200 of forming an injectable amniotic membrane tissue graft;

FIG. 3 is a flow chart showing steps of method 300 of forming an injectable amniotic membrane tissue graft; and

FIG. 4 is a representation of milled amnion 410 suspended in fluid 400.

### DETAILED DESCRIPTION OF EMBODIMENTS OF THE INVENTION

Fetal placental membranes (“PMs”) occupy a unique position in the field of regenerative medicine. This tissue, which derives solely from the developing embryo and fetus, comprises amnion (amniotic membrane or “AM”) and chorion (chorionic membrane or “CM”) fused at a stromal interface and contains a dense concentration of extraembryonic mesenchymal stem cells (“SCs”) in an interstitial matrix rich with multiple classes of biologically active molecules.

The AM is a single layer of epithelial cells—amniocytes—on a relatively thick basement membrane/connective tissue stroma. It derives from the embryonic epiblast, which is adjacent to the primitive streak and contiguous with mesodermal cells giving rise to the notochord, and grows into a fluid-filled sac enveloping the developing embryo and fetus.

The CM is a more complex tissue, adjacent to and invading the maternal uterine wall, but arising from the embryonic trophoblast. In contrast to the histologically simple AM, the chorion is more complex. The trophoblast is a tissue on the uterine surface of the chorion and contains populations and subpopulations of cells. One cell population, the extravillous cytotrophoblast, invades the maternal endometrium. Another population, the syncytiotrophoblast, forms a syncytium of densely nucleated cytoplasm covering the chorionic villi and directly contacting the maternal blood. Like the AM, the CM is also rich in undifferentiated pluripotent extraembryonic mesenchymal stem cells. The antigens giving rise to CM immunogenicity are of both fetal and maternal origin. The maternal antigens are contained in residual bits of decidua (maternal endometrial tissue contacting the placenta) which are typically adherent to the trophoblast of the CM. In addition to antigens of maternal origin, and perhaps more importantly, are the CM fetal antigens. CM tissue components of fetal origin, including connective tissue fibroblasts along with the endothelial cells and residual fetal blood elements contained in fetal blood vessels, can elicit an immunological response in the allograft recipient leading to rejection of the allograft. This rejection can be measured by observing a mixed lymphocyte reaction (“MLR”) in a biopsy specimen. And although the CM stromal layer, which is adjacent to the basement membrane of the AM, contains large/small biomolecules and non-immunogenic SC's expressing only HLA-DR antigens, the trophoblast and fetal connective tissue components express HLA Class I cell surface antigens which may provoke development of a full host immune response to grafted CM. Consequently, intact AM which is manually dissected or “peeled” from the CM at the stromal interface may be used in various allograft preparations whereas use of CM is problematic because of its antigenicity. The CM, however, remains a source of beneficial tissue stroma, including SCs and biomolecules. Therefore, when the placental membranes are received from a volunteer donor and the CM is discarded, at least half of the donor's PM SCs and beneficial biologically active compounds are lost.

AM for tissue graft preparation is potentially available in substantial quantities. There are just under 4 million births per year in the United States, which make up the potential donor pool. From this pool, AM and CM are made available from a suitable subpopulation of donors. Donors undergo a pre-donation screening process to minimize the risk of transmission of maternal or fetal infections agents by way of donated fetal membranes to an eventual tissue graft recipient. This screening procedure includes subjective and objective components. The subjective component includes screening by donor questionnaire for social high-risk factors for infectious disease. Some paid donors are motivated to hide a past social history of high-risk behavior for transmission of sexually transmitted infections, including hepatitis B virus (“HBV”), hepatitis C virus (“HCV”), and human immunodeficiency virus (“HIV”). Accordingly, only volunteer donors are used. The objective (pre-delivery) screening component includes a metabolic panel including liver function studies and assessment of serology for evidence of past or present HBV, HCV, or HIV infection. Routine HLA haplotyping of volunteer donors may be implemented in embodiments of the invention utilizing CM as a tissue graft component. HLA haplotyping of donor and recipient for HLA Class-I and HLA-DR antigens of both donor and recipient may presumably improve the overall survival and engraftment of transplanted viable SCs and other cellular elements present within the tissue graft product while further decreasing the very small, perhaps only theoretical, risk of graft-versus-host disease (“GVHD”) mounted by HLA-mismatched immunocompetent viable maternal T-cells present in the tissue graft product.

Placental membranes from acceptable donors may be excluded by perinatal observations and events. Clinical or laboratory evidence of active maternal or fetal infections around the time of delivery, the most severe example manifest by chorioamnionitis, precludes the use of fetal tissue for graft preparation. Meconium staining of the AF and/or the fetal membranes, although usually not indicative of infection, also eliminates the tissue from the donor pool. Finally, and most commonly, contamination of the placental membranes with a large quantity of maternal blood, feces, or other perinatal sources of gross bacterial or tissue contamination precludes use of the fetal membranes.

Deliveries of placental membranes follow delivery of the infant, and are vaginal or by way of a surgical Cesarean section (“Cesarean”). The use of tissue from donors undergoing a Cesarean delivery largely eliminates gross bacterial contamination of the placental membranes during delivery. Consequently, AM and CM are preferentially procured during a Cesarean, and not a vaginal, delivery. Of the approximately 4 million annual U.S. births mentioned earlier, approximately 33%—1.32 million overall—are by Cesarean delivery. This practice, therefore, reduces the potential donor pool by nearly seventy percent.

Fetal placental membranes suitable for processing and use as tissue grafts may, however, also be collected during a routine vaginal delivery. The bacterial contamination that occurs with vaginal delivery of the placenta is minimal in an uncomplicated delivery, and may be addressed post-partum. Vaginally delivered placentas bearing fetal membranes with no fecal soiling or other source of gross bacterial contamination collected from a vaginally delivered placenta may be effectively treated with sterile washings using topical antibiotic and non-tissue-toxic antimicrobial solutions immediately following delivery and thereafter. Therefore, AM but not AF is potentially available for use as a tissue allograft from between 3.5 and 4.0 million births annually in the U.S. AF is potentially available, with or without AM from the same donor, from between approximately 1.3 and 1.4 million Cesarean deliveries annually in the U.S.

Regardless, AM suitable for use as a tissue allograft is not always available from a Cesarean delivery. Gross contamination rendering the AM unsuitable for tissue grafting may occur during the delivery itself, or later through recognized inadvertent breaks in sterile technique during processing and/or packaging. Additionally, third party quality control testing may reveal microbial contamination.

AM may be collected from suitable volunteer donors and processed for storage prior to use as a tissue allograft in a wide variety of surgical procedures. AM is also used in many non-surgical applications. Some examples of non-surgical uses of an AM tissue graft product include as a biologic dressing/wound covering, a substrate for the creation of artificial skin, and to promote healing of chronically ischemic or infected wounds. Surgical uses of AM tissue grafts include as an adjunct to healing of surgically repaired bone, tendon, other soft tissue, a means to militate the formation of scar tissue and adhesions, and other beneficial applications in surgery and non-surgical minimally invasive medical therapies. AM and AM derivatives are used as biologic dressings containing a source of SCs and growth factors to treat burns, skin pressure ulcers, other chronic open wounds, corneal ulcers, and as a dressing following corneal transplant and other ocular procedures. AM tissue allografts are used to address soft tissue defects and facilitate healing following debridement and repair of damaged cartilage, tendon, bone, nerve, and muscle tissue. AM is under investigation as a connective tissue scaffolding for tissue and organogenesis using extraembryonic SCs and other progenitor cells. Fluidized AM tissue grafts, possessing the anti-inflammatory properties of AM, may be used to prevent the development of postoperative adhesions between the tendon, tendon sheath, and associated tissues following tenolysis, synoviolysis, surgical repair of a damaged tendon, and surgical debridement of necrotic or damaged tendon tissue. Fluidized AM tissue grafts are also useful to mitigate nerve cell death and promote axonal regeneration following early repair of peripheral nerve transections.

An injectable AM tissue graft preparation allows for expanded use of the product in both surgical and minimally invasive settings. The AM tissue graft may be injected into a defined closed space near the end of the surgical procedure, but prior to closing superficial layers of muscle, fascia, and skin at a time when precise placement of the tissue graft under the surgeon's direct visualization is possible. For example, an injectable AM tissue graft, depending on the viscosity of the final product, is delivered by injection though a hypodermic needle as small as 30-gauge (“G”) into a closed tendon sheath following tenolysis or tendon repair, into a closed joint capsule following repair of intra-articular cartilage, ligaments, or total joint replacement, into the peritoneal cavity following closure of the abdominal wall, into the pleural space following closure of the chest wall, and into the subdural space following closure of the spinal or intracranial dura mater. An injectable AM tissue graft of higher viscosity is injected through a 23G, 22G, 21G, 20G, 18G, 16G, or larger-bore hypodermic needle in these and other surgical and minimally invasive applications. An injectable AM tissue graft of lower viscosity is injected through a 25G or 30G needle for use in fine neural repair, aesthetic surgery, and other applications. Following wound closure, an injectable AM tissue graft may also be re-injected into the defined closed space during the perioperative and postoperative period if deemed useful by the surgeon or other healthcare provider.

An injectable AM tissue graft may also be injected into a tissue bed in a minimally invasive non-surgical setting. A syringe containing a quantity of AM tissue graft is fitted with a hypodermic needle of suitable size for the intended application. The needle is directed to the target tissue bed using visualization and palpation of external landmarks by the provider. Placement of the needle within the target tissue space or tissue may, in some embodiments, be facilitated with fluoroscopy or other non-invasive and minimally invasive imaging modalities. Some example uses of the injectable AM tissue allograft include intra-articular injection for treatment of injured ligaments, cartilage, and bone; intra-capsular injection of tendon injuries, synovitis, tenosynovitis, and other inflammatory joint conditions; intra-thecal injection for treatment of spinal cord and brain injuries, aseptic meningitis, and other central neurological infections and inflammatory conditions; and other minimally invasive non-surgical applications.

In all of these and other applications, there is strong evidence that the presence of the aforementioned active biomolecules and other factors present in the AM tissue graft improves healing across a broad range of tissue types, locations within the body, and applications. Reporting of clinical results may eventually lead to the use of AM tissue grafts as a standard therapy, and possibly even the accepted best practice, for the treatment of a variety of conditions.

Preparation and sterilization of AM for later use as a tissue allograft, in some embodiments of the invention, includes drying, packaging, sterilization, and storage. Drying discourages bacterial growth and helps maintain sterility during storage. Drying facilitates standardization of the final AM tissue graft in terms of weight per unit volume of dried AM prepared under standardized parameters of temperature, humidity, and time. Drying, however, has negative effects on AM and is not always used in the preparation of AM tissue grafts. Drying may be accomplished by heating or freezing in a partial vacuum (lyophilization or “freeze drying”) to minimize water-ice crystal formation and cellular disruption. Although some viable SCs are preserved by drying under controlled conditions, other SC's die during processing. It is not fully known how drying and storage affect the concentration of the biologically active non-cellular components of AM, though a significant decrease in concentration of intact proteins and other large biomolecules is possible. Sterilization by head or radiation destroys the cellular components of AM preparations, including SCs. Thermal or irradiative sterilization methods may also denature proteins and alter or destroy other large biologically active molecules. Some allograft preparations reconstitute the dried AM using a tissue preservative solution prior to packaging and storage. The medium used to reconstitute the dried AM is typically a buffered isotonic solution containing water and electrolytes, but no growth factors, other active biomolecules, or additional SCs. And although the dimensions and weight of dried AM may be easily measured and recorded in the available graft tissue, the absolute number and concentration of viable SCs per unit weight or volume of tissue, which may prove to have high clinical relevance for optimal dosing, is not known by the patient-treating provider.

It is useful to employ AM tissue graft preparations of varying viscosity for transplantation with knowledge of expected results based upon reproducibility. Variations in viscosity affect the tendency of the AM tissue graft to remain and engraft at the site of placement. Differences in viscosity are considered based upon the intended use of the standardized AM tissue graft. Generally, standardized AM tissue grafts are prepared in three reproducible, standardized viscosities: high viscosity; medium viscosity; and low viscosity.

High-viscosity standardized AM tissue graft has a concentration of ground AM of greater than 10 mg/ml, with or without an additional biologically compatible “thickening agent.” Some examples of applications where a high-viscosity standardized AM tissue graft may be used include the non-invasive or minimally-invasive treatment of entero-cutaneous, entero-vaginal, entero-enteric, broncho-pleural, tracheal-esophageal fistulas; graft-repair of osteochondral defects in the knee, hop, ankle, wrist, hand, and other joints; microfractures and small facial fractures; and filling of large bone tissue void following surgical treatment of certain cancers.

Medium-viscosity standardized AM tissue graft has a concentration of ground AM of between 1 mg/ml and 10 mg/ml. Examples of applications where a medium-viscosity standardized AM tissue graft may be used include treatment of wound sinus tracts, grafting of cutaneous and soft-tissue defects resulting from deep thermal or radiation burns; spinal and other bony fusion procedures (when combined with currently available bone putty or as a stand-alone application into a cervical or lumbar intervertebral spacer); facial trauma and facial fracture treatment; bone grafting; alveolar cleft (“cleft palate”) grafting; treatment of dental/tooth tissue defects; chronic inflammatory bursitis; intervertebral facet-based pain; tears of the meniscal cartilage; application to entero-entero and other surgical anastomoses; treatment of non-union and mal-union of fractures, intra-peritoneal application following surgical adhesiolysis; intra-peritenon implantation following Achilles' tendon debridement and anastamotic repair; defects of the calvarium following trauma; emergency decompressive craniotomy; surgical breast reconstruction; and following acetabular and other articular joint surface resurfacing.

Low-viscosity standardized AM tissue graft has a concentration of ground AM of less than 1 mg/ml. Examples of applications where a low-viscosity standardized AM tissue graft may be used include treatment of chronic wounds, radiation burns, and thermal injury by subcutaneous injection; injection into peri-rotator cuff soft tissues following rotator cuff repair; injection to facilitate non-surgical repair and healing of supraspinatus, infraspinatus, teres minor, and subscapularis tears; other muscle, ligament, tendon, and soft-tissue tears; epicondylitis; and other similarly debilitating chronic fascial inflammatory conditions such as plantar fasciitis or fasciolosis.

Substantial differences in both the absolute amount and concentration per unit volume of biologically active substances in the final preparation arise in currently available preparations based upon the preparation methods used. Existing AM tissue graft preparations are typically formed by suspending a single ground amnion in a suitable fluid. The weight of an individual amnion, however, is variable. Although recording placental weights may not directly reflect amnion weights, a published study (Lurie, et al. (1999) “Human fetal-placental weight ratio in normal singleton near-term pregnancies” Gynecologic and Obstetric Investigation, 48(3): 155-57) of 431 uncomplicated singleton deliveries revealed a mean placental weight of 613+/−123.8 mg, ranging from 319 mg to 1,266 mg. Thus, the weight of the human placenta and its constituent components commonly ranges by nearly 40% around the mean weight and may vary by as much as 400%. The commonly used techniques in preparation of AM-derived suspensions, therefore, result in a preparation with a completely arbitrary total amount and concentration of AM. In some preparation methods, AM is subject to different degrees of drying, whether intentionally or unintentionally. Random samples of AM processed and stored in non-standardized conditions with respect to temperature and drying time revealed an average weight of 1.02+/−0.12 mg/cm².

What is lacking in the prior art, therefore, is an injectable liquid AM-derived tissue graft preparation incorporating an effective concentration of active biomolecules available from an individual donor or the largest possible pool of volunteer donors.

Embodiments of this invention address these fundamental AM tissue graft requirements—high concentration of beneficial biomolecules in a standardized preparation with no antigenic material and minimal waste of available donor tissue—by forming a liquid, reconstituted tissue graft preparation from a dried, milled particulate AM rehydrated in a suspension with a suitable fluid at a suitable viscosity for use in surgical and minimally invasive applications requiring an injectable tissue graft preparation.

Disclosed is an injectable AM tissue graft preparation, including a method of forming same. The AM tissue graft comprises milled AM reconstituted with a suitable fluid. The preparation is used by medical providers as a tissue graft. In some embodiments, the tissue graft is delivered to the host tissue by intraoperative application or injection, non-operative percutaneous injection, or direct application to injured, ischemic, infected, inflamed, surgically manipulated, or otherwise damaged tissue. The preparation is also used, in some embodiments, by laboratory researchers as a stable source of material for basic science research of the effects of AM preparations on healthy, diseased, and damaged tissue in the field of orthopedics, neurology, neurosurgery, general surgery, gynecologic surgery, regenerative medicine and in other medical and scientific disciplines. The use of milled, AM reconstituted in a suitable fluid, such as a buffered isotonic electrolyte solution and/or cryoprotectant, for example, maximizes delivery of a wide range of beneficial biologic substances within a non-antigenic liquid tissue graft to the recipient tissue/treatment site.

In various embodiments of the invention, the viscosity and size of the amniotic membrane particles comprising the tissue graft preparation will vary according to the intended application of the tissue graft. For instance, tissue graft preparations prepared for application to soft tissue, e.g., muscle, tendon, ligament, or skin, will include amniotic membrane particles larger than 75 microns, between 75 microns and 100 microns, between 85 microns and 110 microns, between 95 microns and 120 microns, or between 100 microns and 130 microns. Tissue graft preparations for soft tissue applications will have a final amniotic particle concentration of between 0.05 g/ml and 0.10 g/ml, between 0.01 g/ml and 0.05 g/ml, or between 0.10 g/ml and 0.15 g/ml. Alternatively, tissue graft preparations prepared for application to bone will include amniotic membrane particles smaller than 75 microns, between 60 microns and 75 microns, between 50 microns and 60 microns, between 40 microns and 50 microns, between 30 microns and 40 microns, between 20 microns and 30 microns, between 10 microns and 20 microns, between 1 micron and 10 microns, between 0.1 microns and 1 micron, between 0.01 microns and 1 micron, between 1 micron and 25 microns, between 25 microns and 50 microns, or between 50 microns and 75 microns. Tissue graft preparations for bone applications will have a final amniotic particle concentration of between 0.15 g/ml and 0.20 g/ml, between 0.20 g/ml and 0.25 g/ml, or between 0.25 g/ml and 0.30 g/ml.

Furthermore, in tissue graft preparations designed for application to bone, hydroxyapatite will be added to the tissue graft. For instance, hydroxyapatite may be added at a final concentration of about 1%, about 2%, about 2.5%, about 4%, about 5%, about 7.5%, about 10%, about 12.5%, or about 15%. Hydroxyapatite may also be added at a final concentration of 1%, 2%, 2.5%, 4%, 5%, 7.5%, 10%, 12.5%, or 15%.

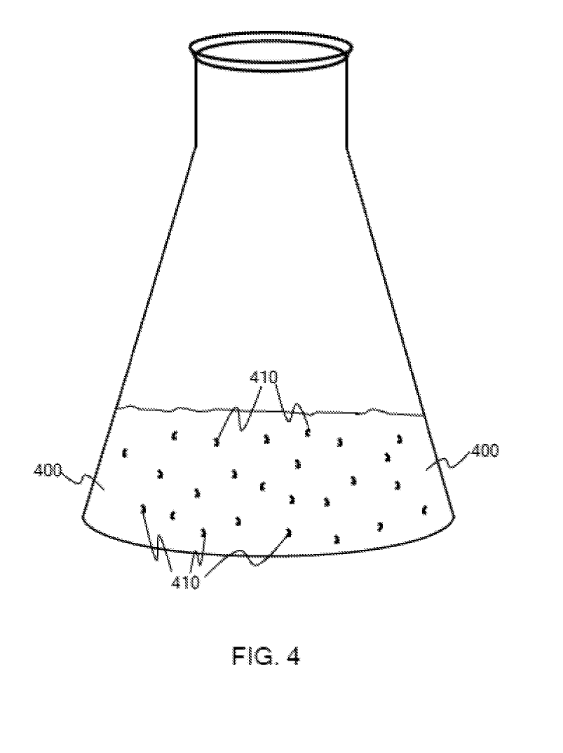

FIG. 4 shows an injectable AM tissue graft 450 comprising a milled AM 410 suspended in a fluid 400. Details regarding the composition and preparation of the injectable AM tissue graft 450 are provided below and throughout this disclosure.

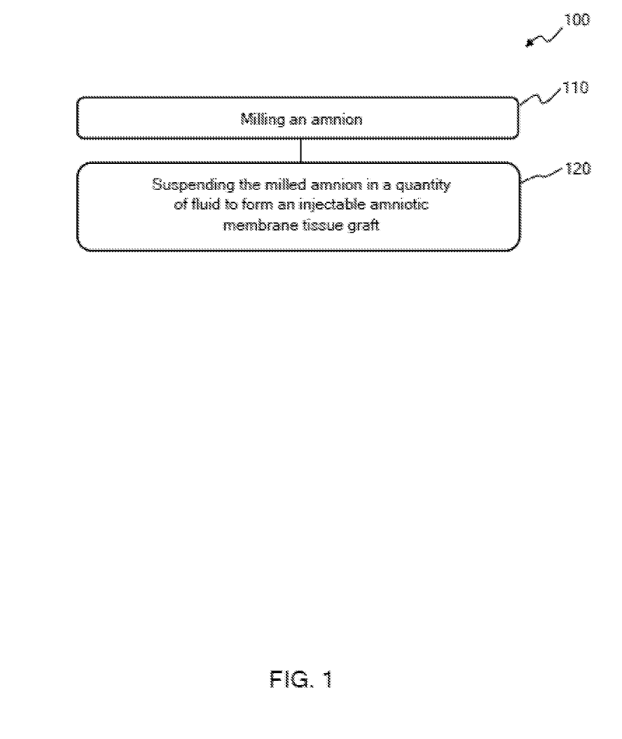

FIG. 1 shows a method 100 of forming an injectable AM tissue graft 450 in some embodiments of the invention. Method 100 requires an amnion. In some embodiments of the invention, the AM comes from a volunteer human donor. Accepting amniotic tissue from volunteer donors and excluding non-volunteer and/or paid donors from the donor pool is consistent with internationally well-established tissue donation protocols because it reduces the chance that an infectious agent present in the donor will be transmitted to the graft recipient, resulting in an infection in the recipient. Screening of potential volunteer donors, therefore, includes obtaining a comprehensive past medical and social history, complete blood count, liver and metabolic profile, and serologic testing for HBV, HCV, and HIV, in some embodiments.

In some embodiments, donor tissue is obtained during delivery by elective Cesarean section. The use of a Cesarean-delivered AM to prepare the injectable AM tissue graft 450 is preferable in some embodiments because an AM delivered by Cesarean section is obtained and packaged under strict sterile technique in the operating room, with essentially no/minimal microbial contamination. Following Cesarean delivery of the infant, the placenta is delivered. Operating room personnel familiar with sterile technique and tissue handling perform all steps necessary to prepare the tissue for packaging. The combined fetal membranes (AM and CM) are dissected from the maternal placental plate (decidua). The combined fetal membranes are gently washed with sterile 0.9% saline solution to remove all visible traces of maternal blood, AF, and any other visible, potentially contaminating material. The dissected and washed combined fetal membranes are then placed in a sterile specimen container and a quantity of 0.9% sterile saline is added sufficient to completely submerge the combined fetal membranes. The sterile container containing the fetal placental membranes collected under sterile conditions in the operating room are then securely closed and placed in a donor tissue specimen bag. This first bag is then placed within a second bag, which is then sealed, labeled, and taken from the operating room for packaging in an insulated ice-bath container. A patient data sheet containing information regarding the maternal donor is placed in the container, and a separate copy of this information is recorded and logged prior to closing the package. The packaged specimen container is then immediately transported to the processing facility by staff who rotate on call, such that there is minimal delay following delivery before the donor tissue arrives at the separate facility for processing.

Despite the preference for a Cesarean-delivered AM in order to increase the pool of potential donors and other of the aforementioned reasons, vaginally delivered fetal membranes are utilized in some embodiments. Great care must be afforded the vaginally-delivered placental tissue to prevent microbial contamination. Vaginally-delivered fetal membranes are not acceptable donor tissue if there is fecal or other grossly visible contamination, or if there is contact of the placental membranes with clothing, bedding, non-sterile unprepped skin, or other non-sterile surfaces during delivery or prior to sterile packaging. Neither a vaginally-delivered AM nor a Cesarean-delivered AM is acceptable donor tissue if there is visible staining of the fetal membranes with meconium. Following delivery, the steps for preparing vaginally delivered fetal membranes are the same as the above description of preparing Cesarean-delivered fetal membranes. A fully gowned-and-gloved staff member processes the fetal membranes on a sterile field established on a back table, or similar surface, in the labor/delivery room. An additional step comprising rinsing the vaginally delivered fetal membranes with an antimicrobial solution is used in some embodiments. After washing with 0.9% sterile saline, the vaginally delivered dissected fetal membranes are washed with a topical antimicrobial solution. Examples of the topical antimicrobial solution used to wash the vaginally delivered fetal membranes, in some embodiments, are a 0.5% aqueous solution of glutaraldehyde (which is then washed off the donor tissue using a final rinse of 0.9% sterile saline prior to packaging), a Penicillin-Streptomycin solution comprising 50-100 International Units (“IU”) per ml of penicillin and 50-100 micrograms/ml of Streptomycin, or a 0.0125% aqueous solution of sodium hypochlorite. These examples are not meant to be limiting. Other antimicrobial solutions toxic to infectious microorganisms at non-cytotoxic concentrations may also be used. The fetal membranes, following the antimicrobial washing, are then placed in a sterile specimen container, covered with 0.9% sterile saline solution, and sealed in sequential sterile bags as described above for Cesarean-delivered fetal membranes. The prepared, sealed, labeled, recorded, and packaged donor fetal membranes are then delivered to the separate tissue processing facility, as described above.

Immediately upon receipt at the processing facility, the shipping label is examined and information regarding the specimen and donor is recorded. The shipping container is examined for integrity, including confirmation of an intact tamper-proof seal. The shipping container is then opened and the inner bag containing the fetal membranes and amniotic fluid is examined. An infrared temperature sensor is directed at the tissue bag to confirm a temperature of between 6 and 10 degrees Celsius. If there is any indication of damage to the outer container, the inner bag containing the placental membranes is examined with particular care. If damage to the inner bag is identified or the tamper-proof seal is broken or damaged, the specimen is not used to prepare the injectable AM tissue graft. A donor/specimen data sheet within the container is then reviewed to validate the donor's credentials. The information on the data sheet is compared to the donor ID on the specimen bag to confirm the data sheet for the donor matches the specimen. This information is recorded and included in the permanent batch record for that specific donor. These credentials include donor lot numbers and expiration dates. All validation dates and times are confirmed. A donor tissue specimen that is unacceptable for any reason is discarded. The date, time, and hospital from which the donor specimen was received is recorded. The outside of the bag containing the two separate sterile specimen containers is then sprayed with isopropyl alcohol and manually wiped down. The logged and cleaned specimen bag containing the donor placental membranes is then stored in a locked refrigerator in an ice water bath, but not frozen.

Following receipt of the donor tissue, the amnion is cleaned and prepared for milling, as practiced in some embodiments shown in FIG. 1. Under strict sterile technique, the specimen bag is opened using sterile scissors and the donor specimen comprising placental membranes is carefully poured into a large sterile basin. Using sterile forceps, the AM is peeled from the CM, which separates at the AM basement membrane/CM stromal interface. The AM is placed on a sterile cutting board, CM-side facing up. The CM side is gently wiped with sterile cloth towels, taking care to remove any adherent bits of CM and clotted blood which may not have been completely rinsed from the AM immediately following the delivery prior to packaging. Both sides of the AM are once again washed with sterile 0.9% saline and rinsed with an antimicrobial solution in some embodiments, such as 0.5% aqueous solution of glutaraldehyde for example.

As shown in FIG. 1 the first step 110 of method 100 is milling an amnion. In some embodiments, for example, the AM is placed in a temperature-controlled ball-grinding mill (i.e. “CryoMill” for cryogenic grinding, manufactured by Retsch Corporation, Haan, Germany). In some embodiments, the grinding jar and milling balls are weighed prior to placement of a quantity of AM in the grinding jar. After placement in the grinding jar, the dried AM is pre-cooled in a liquid nitrogen bath to minus 196° Celsius and then ground for approximately 4 minutes. This process results in an AM particle size of 5 microns. The grinding jar is again weighed, and the weight of milled AM 410 contained within is determined. Determination of the weight of milled AM 410 allows for standardization of the injectable AM tissue graft 450, as provided in some embodiments of the invention. These examples are not meant to be limiting. The milling process may be longer or shorter than 4 minutes and the milled AM 410 particle size may be larger or smaller than 5 microns, depending on the desired viscosity, final concentration of AM/unit volume in the tissue graft, and other factors desired by the surgeon or other end-user health care provider.

Also shown in FIG. 1, step 120 of method 100, in some embodiments, comprises suspending the milled AM 410 in a quantity of fluid 400 to form an injectable AM tissue graft. In some embodiments, the milling jar containing the milled particulate AM is opened and the milled AM 410 is washed from the jar and balls using a measured quantity, usually 50 cc's or less for example, of a fluid 400. In some embodiments, the fluid 400 is a buffered isotonic solution (an example is “Plasma-Lyte A,” manufactured by Baxter International, Inc., Deerfield, Ill.). In some embodiments, the fluid 400 is a cryoprotectant (an example is CryoStor CS-10, a 10% solution of dimethylsulfoxide (“DMSO”), manufactured by BioLife Solutions, Inc., Bothel, Wash.). These examples are not meant to be limiting, other examples of non-cytotoxic fluids may be used. In some embodiments, the milling jar is weighed prior to opening. In some embodiments, a standard quantity of fluid 400, 50 cc's for example, is added to liquefy and reconstitute the milled AM 410. In some embodiments, the reconstituted AM (“AMPL”) has a known weight of AM per volume of AMPL. The formed injectable AM tissue graft 450 comprises the reconstituted AMPL.

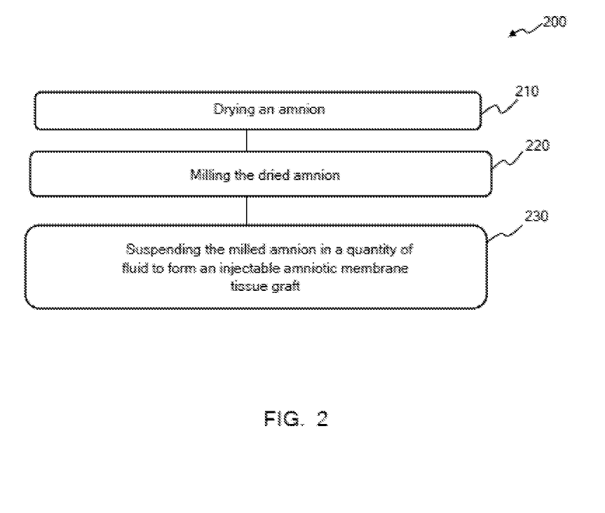

FIG. 2 shows a method 200 of forming the injectable AM tissue graft 450 in some embodiments of the invention. Step 210 comprises drying an amnion, as is practiced in some embodiments. Following the dissecting and cleaning steps described above but prior to the milling step 110 of method 100, the AM is further prepared and dried. Using sterile scissors, the cleaned and treated AM from the initial processing is cut into pieces, placed on a mesh, and then the mesh-AM is placed on a rack for drying. In some embodiments, the AM is dried under standardized conditions of temperature, humidity, and time. In some embodiments, the AM is simply dried in a sterile enclosure under ambient conditions until brittle.

Step 220 of method 200 shown by FIG. 2 comprising milling the dried amnion, as described above. The following step 230 comprises suspending the milled AM 410 in a quantity of fluid 400 to form an injectable AM tissue graft, also as described above.

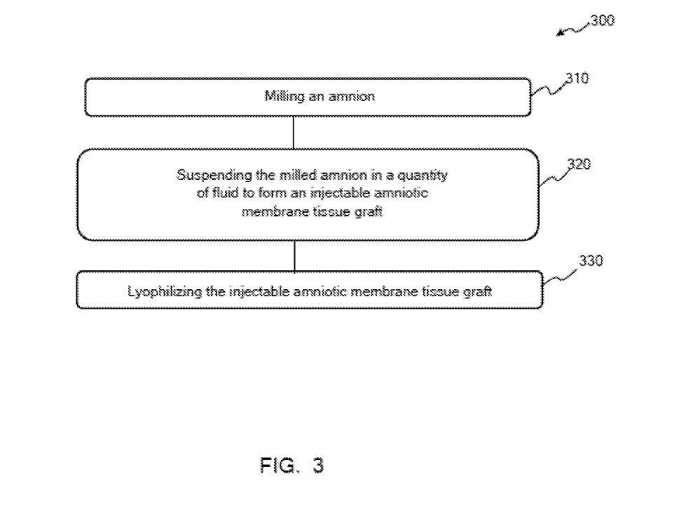

FIG. 3 shows a method 300 of forming the injectable AM tissue graft 450 in some embodiments of the invention. Step 310 of method 300 comprises milling an amnion. In some embodiments, the amnion is obtained and initially processed in the aforementioned manner. Step 320 of method 300 comprises suspending the milled AM 410 in a quantity of fluid 400 to form an injectable AM tissue graft. Step 330 of method 300, in some embodiments, comprises lyophilizing the injectable AM tissue graft. In some embodiments, the completed injectable AM tissue graft 450 formed following step 320 is pipetted into empty product vials and placed in a lyophilization unit for controlled removal of water and other volatiles prior to final packaging and shipping. Many commercially marked lyophilization units are widely available and their use is known to those with skill in the art. The packaging vials of lyophilized allograft are then sterilely sealed, labeled, and cooled in a controlled-rate freezer to minus 80° Celsius, in some embodiments. The vials are then maintained at minus 80° Celsius until needed for use.

When needed for use, the lyophilized contents in the sealed vial are warmed under ambient conditions to a temperature above 0° Celsius. A quantity of buffered isotonic solution is injected into the sealed vial to reconstitute the lyophilized AM tissue graft. In some embodiments, the quantity is just enough fluid 400 to re-hydrate and suspend the AM particles. In some embodiments, the quantity of buffered isotonic solution is adjusted as necessary to obtain an injectable AM tissue graft of appropriate viscosity for injection through a 25G hypodermic needle bore-size. In some embodiments, less buffered isotonic solution may be used to create an AM tissue graft which may be injected through a larger-bore hypodermic needle, or for direct topical application, as required by the medical provider.

In some embodiments of method 100 shown in FIG. 1 and method 200 shown in FIG. 2, a small quantity of the formed injectable AM tissue graft, approximately 0.5 ml, for example, is drawn into a sterile 2 cc syringe and extruded through a 25 gauge needle to ensure the allograft is sufficiently fluid to be percutaneously or intraoperatively injected into the recipient tissue bed. In some embodiments, the viscosity of the injectable AM tissue graft 450 is adjusted by mixing an additional measured quantity of buffered isotonic solution with the injectable AM tissue graft, and recording the final concentration of AM and SC per ml accordingly. In some embodiments, the final concentration of AM and/or SC per ml is adjusted with additional buffered isotonic solution to an end-user's pre-ordered concentration requirement, based upon the intended use of the completed allograft. None of the aforementioned examples of mixing the formed injectable AM tissue graft 450 with various quantities of buffered isotonic solution are meant to be limiting, they are merely given as useful examples.

The embodiments and examples set forth herein were presented in order to best explain the present invention and its practical application, and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above, and are intended to fall within the scope of the appended claims.

1. A tissue graft comprising a suspension of amniotic membrane particles, wherein the average amniotic membrane particle size is less than 75 microns and the concentration of amniotic membrane particles in the suspension is 0.15-0.20 g/mL.

2. A tissue graft comprising a suspension of amniotic membrane particles, wherein the average amniotic membrane particle size is between about 75 and about 100 microns and the concentration of amniotic membrane particles in the suspension is 0.05-0.10 g/mL.

3. The tissue graft of claim 1 or 2, wherein the suspension further comprises hydroxyapatite.

4. The tissue graft of claim 1, wherein the tissue graft is substantially free of chorion particles.

5. A tissue graft comprising a suspension of amniotic membrane particles at a concentration greater than 10 mg/ml.

6. A tissue graft comprising a suspension of amniotic membrane particles at a concentration between about 1 mg/ml and about 10 mg/ml.

7. A tissue graft comprising a suspension of amniotic membrane particles at a concentration less than 1 mg/ml.

8. A tissue graft according to any one of claims 5-7, further comprising a thickening agent.

9. A method of applying to a subject a tissue graft comprising a suspension of amniotic membrane particles, the method comprising:
applying a tissue graft to a location in or on the subject, wherein the suspension has a concentration, a viscosity or an average amniotic membrane particle size selected to enhance the applicability of the tissue graft at the location.

10. The method of claim 9, wherein the location comprises bone.

11. The method of claim 10, wherein the average amniotic membrane particle size is less than 75 microns.

12. The method of claim 9, wherein the average amniotic particle size is at least 75 microns.

13. The method of claim 9, wherein the average amniotic particle size is between about 75 and about 100 microns.

14. The method of claim 9, wherein the concentration of amniotic membrane particles in the suspension is 0.05-0.10 g/mL.

15. The method of claim 9, wherein the concentration of amniotic membrane particles in the suspension is 0.10-0.20 g/mL.

16. The method of claim 9, wherein the tissue graft is substantially free of chorion particles.

17. The method of claim 10, wherein the suspension further comprises hydroxyapatite.

18. The method of claim 9, wherein the location comprises soft tissue.

19. The method of claim 18, wherein the soft tissue is selected from the group consisting of muscle, tendon, ligament, and skin.
